(19) United States
(12) Patent Application Publication
(10) Pub. No.: US 2009/0247732 A1
(43) Pub. Date: Oct. 1, 2009

(54) Synthesis of glycopeptides with superior pharmacokinetic properties

(75) Inventors: Jonathan Duffield, San Diego, CA (US); Kenneth Marby, San Diego, CA (US); Sean O'Hare, Carlsbad, CA (US); Chan-Kou Hwang, San Diego, CA (US); Yoshitaka Ichikawa, San Diego, CA (US)

Correspondence Address: CATALYST LAW GROUP, APC, 9710 SCRANTON ROAD, SUITE S-170, SAN DIEGO, CA, 92121, US

(73) Assignee: Optimer Pharmaceuticals, Inc., San Diego, CA (US)

(21) Appl. No.: 10/578,603

(22) PCT Filed: Nov. 1, 2004
(86) PCT No.: PCT/US04/36551

§ 371 Date: May 4, 2006

Related U.S. Application Data
(60) Provisional application No. 60/516,838, filed on Nov. 4, 2003.
(60) Provisional application No. 60/557,631, filed on Mar. 30, 2004.
(60) Provisional application No. 60/598,215, filed on Aug. 2, 2004.

(51) Int. Cl. C07K 9/00; C07K 1/113

(52) U.S. Cl. 530/322; 530/333

(57) ABSTRACT

The present invention relates to novel glycopeptides having improved pharmacokinetic properties, improved stability, methods of preparing said novel glycopeptides, and methods to use said novel glycopeptides.

### RELATED APPLICATIONS

The present application is related to, and claims priority from, U.S. Provisional Patent Application No. 60/516,838, filed Nov. 4, 2003, U.S. Provisional Patent Application No. 60/557,631, filed Mar. 30, 2004, and U.S. Provisional Patent Application No. 60/598,215, filed Aug. 2, 2004, the entire disclosures of which are herein incorporated by reference.

### FIELD OF INVENTION

The present invention relates generally to novel glycopeptides having improved pharmacokinetic properties, methods of producing the same, and methods of using the same. The present invention also relates to novel glycopeptides in which a particular sugar motif can serve as a stable surrogate for a specific amino acid residue.

### BACKGROUND OF THE INVENTION

Carbohydrates play important roles in biological processes and the saccharide motifs of glycopeptides and glycoproteins are critical determinants of conformational and physicochemical properties. The number of peptide-based drugs and clinical candidates in the field of medicine is growing steadily, with new applications emerging in cardiovascular disease, metabolic disorders, cancer, anti-bacterial, and anti-viral therapeutic areas among many others. But the potential of peptides as therapeutic agents is limited by their significant instability in the presence of peptidase enzymes, which drastically reduces the drugs' half-lives in vivo. There are only limited synthetic approaches with which it is possible to modify the pharmacokinetic properties of peptides, typically using preassembled, protected glycosyl-amino acids via solid-phase peptide synthesis (Powell, M. F. et al., Pharm. Res. 1993, 10, 1268-1273), (Polt, R. J. Am. Chem. Soc. 2003, 125, 5823-5831). To date, the prior art does not provide for a method to effectively address this significant need. The present invention provides for a solution to meet this and other needs.

### SUMMARY OF THE INVENTION

The present invention relates to novel glycopeptides, methods for preparing novel glycopeptides, and for using said novel glycopeptides to enhance the stability of a peptide towards peptidase enzymes. Unlike the prior art, the present invention provides for the conjugation of carbohydrates with native peptides and peptide surrogates. Therefore, the present invention advantageously confers highly desirable drug-like properties upon the resulting glycoconjugate.

### BRIEF DESCRIPTION OF THE FIGURES

FIG. 1 shows Group A saccharide and disaccharide group epitopes.

FIG. 2 shows Group B saccharide and disaccharide group epitopes.

FIG. 3 shows Group C saccharide and disaccharide group epitopes.

FIG. 4 shows Group D saccharide and disaccharide group epitopes.

### DETAILED DESCRIPTION OF THE INVENTiON

The present invention generally relates to a novel methodology for the addition of one or more carbohydrate moieties to peptides through a linker or spacer or directly without such a linker or spacer between the anomeric position of the carbohydrate and an amino, hydroxyl or carboxyl group on the peptide, thereby significantly increasing the half-life of said peptide in a biological system.

To the best of our knowledge, there is no precedent in literature or prior-art, which teaches that a particular sugar motif can serve as a stable surrogate of a specific amino acid residue. The structures of the carbohydrate moieties (sugar motifs) vary from natural neutral-, amino-, and acidic-sugars to those with five- or six-membered sugar frameworks with a wide variety of functional groups that are combinatorially generated.

Specifically, the introduced sugar motif can replace the serine residue if the sugar motif has a hydroxyl group, or replace basic amino acid residues, including lysine, arginine, or histidine, if the sugar motif contains a basic functionality such as an amino group.

### DEFINITIONS

The compounds of the invention comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of diastereomers, as well as single diastereomers of the compounds of the invention are included in the present invention. The terms “S” and “R” configuration, as used herein, are as defined by the IUPAC 1974 Recommendations for Section E, Fundamental Stereochemistry, Pure Appl. Chem. (1976) 45, 13-30.

The compositions containing the compound(s) of the invention can be administered for prophylactic and/or therapeutic treatments. An amount adequate to accomplish this is defined as “therapeutically effective amount or dose.” Amounts effective for this use will depend on the severity and course of the disease or condition, previous therapy, the patient's health status and response to the drugs, and the judgment of the treating physician. In prophylactic applications, compositions containing the compounds of the invention are administered to a patient susceptible to or otherwise at risk of a particular disease or condition. Such an amount is defined to be a “prophylactically effective amount or dose.” In this use, the precise amounts again depend on the patient's state of health, weight, and the like.

Once improvement of the patient's conditions has occurred, a maintenance dose is administered if necessary. Subsequently, the dosage or the frequency of administration, or both, can be reduced, as a function of the symptoms, to a level at which the improved condition is retained. When the symptoms have been alleviated to the desired level, treatment can cease. Patients can, however, require intermittent treatment on a long-term basis upon any recurrence of the disease symptoms.

In general, a suitable effective dose of the compound of the invention will be in the range of 0.1 to 1000 milligrams (mg) per recipient per day, preferably in the range of 1 to 100 mg per day. The desired dosage is preferably presented in one, two, three, four or more subdoses administered at appropriate intervals throughout the day. These subdoses can be administered as unit dosage forms, for example, containing 5 to 1000 mg, preferably 10 to 100 mg of active ingredient per unit dosage form. Preferably, the compounds of the invention will be administered in amounts of between about 1.0 mg/kg to 250 mg/kg of patient body weight, between about one to four times per day.

A “pharmacological composition” refers to a mixture of one or more of the compounds described herein, or physiologically acceptable salts thereof, with other chemical components, such as physiologically acceptable carriers and/or excipients. The purpose of a pharmacological composition is to facilitate administration of a compound to an organism.

The term “pharmaceutically acceptable salts” of the compounds of the invention include those derived from pharmaceutically acceptable inorganic and organic acids and bases. Examples of suitable acids include hydrochloric, hydrobromic, sulfuric, nitric, perchioric, fumanc, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-D-gluconic acid, and the like. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N(C₁-C₄)₄⁺ salts, and the like. Illustrative examples of some of these include sodium hydroxide, potassium hydroxide, choline hydroxide, sodium carbonate, and the like. The term “carbon chain” may embrace any alkyl, alkenyl, alkynyl, or heteroalkyl, heteroalkenyl, or heteroalkynyl group, and may be linear, cyclic, or any combination thereof. If part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining chain length. If the chain contains heteroatoms as part of the backbone, those atoms are not calculated as part of the carbon chain length.

The term “physiologically acceptable carrier” refers to a carrier or diluent that does not cause significant irritation to an organism and does not abrogate the biological activity and properties of the administered compound.

The term “excipient” refers to an inert substance added to a pharmacological composition to further facilitate administration of a compound. Examples of excipients include but are not limited to, calcium carbonate, calcium phosphate, various sugars and types of starch, cellulose derivatives, gelatin, vegetable oils and polyethylene glycols.

The term “alkyl”, alone or in combination, refers to an optionally substituted straight-chain, optionally substituted branched-chain, or optionally substituted cyclic alkyl radical having from 1 to about 30 carbons (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀), preferably 1 to 12 carbons (e.g., C₁, C₂, C₃, C₄, C₅, C₆, C₇, C₈, C₉, C₁₀, C₁₁, C₁₂). Examples of alkyl radicals include methyl, ethyl, n-propyl, isopropyl, n-butyl, isobutyl, sec-butyl, tert-butyl, tert-amyl, pentyl, hexyl, heptyl, octyl and the like.

The term “cycloalkyl” embraces cyclic configurations, is subsumed within the definition of alkyl and specifically refers to a monocyclic, bicyclic, tricyclic, and higher multicyclic alkyl radicals wherein each cyclic moiety has from 3 to about 8 carbon atoms (e.g., C₃, C₄, C₅, C₆, C₇, C₈). Examples of cycloalkyl radicals include cyclopropyl, cyclobutyl, cyclopentyl, cyclohexyl and the like.

A “lower alkyl” is a shorter alkyl, e.g., one containing from 1 to about 6 carbon atoms (e.g., C₁, C₂, C₃, C₄, C₅, C₆).

The term “alkenyl,” alone or in combination, refers to an optionally substituted straight-chain, optionally substituted branched-chain, or optionally substituted cyclic alkenyl hydrocarbon radical having one or more carbon-carbon double-bonds and having from 2 to about 30 carbon atoms (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C2₁, C₂₂, C₂₃, C₂₄, C₂₅, C₂₆, C₂₇, C₂₈, C₂₉, C₃₀), more preferably 2 to about 18 carbons (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈). Examples of alkenyl radicals include ethenyl, propenyl, butenyl, 1,4-butadienyl and the like. The term can also embrace cyclic alkenyl structures.

The term “lower alkenyl” refers to an alkenyl having from 2 to about 6 carbons (e.g., C₂, C₃, C₄, C₅, C₆).

The term “alkynyl,” alone or in,combination, refers to an optionally substituted straight-chain, optionally substituted branched-chain, or cyclic alkynyl hydrocarbon radical having one or more carbon-carbon triple-bonds and having from 2 to about 30 carbon atoms (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀), more preferably 2 to about 12 carbon atoms (e.g., C₁, C₂, C₃, C₄, C₅, C₆, C₇, C₈, C₉, C₁₀, C₁₁, C₁₂). The term also includes optionally substituted straight-chain or optionally substituted branched-chain hydrocarbon radicals having one or more carbon-carbon triple bonds and having from 2 to about 6 carbon atoms (e.g., C₂, C₃, C₄, C₅, C₆) as well as those having from 2 to about 4 carbon atoms (e.g., C₂, C₃, C₄). Examples of alkynyl radicals include ethynyl, propynyl, butynyl and the like.

The terms “heteroalkyl,” “heteroalkenyl” and “heteroalkynyl” include optionally substituted alkyl, alkenyl and alkynyl structures, as described above, and which have one or more skeletal chain atoms selected from an atom other than carbon, e.g., oxygen, nitrogen, sulfur, phosphorous or combinations thereof.

The term “alkoxy,” alone or in combination, refers to an alkyl ether radical, alkyl-O-, wherein the term alkyl is defined as above. Examples of alkoxy radicals 25 include methoxy, ethoxy, n-propoxy, isopropoxy, n-butoxy, iso-butoxy, sec-butoxy, tert-butoxy and the like.

The term “aryloxy,” alone or in combination, refers to an aryl ether radical wherein the term aryl is defined as below. Examples of aryloxy radicals include phenoxy, benzyloxy and the like.

The term “alkylthio,” alone or in combination, refers to an alkyl thio radical, alkyl-S-, wherein the term alkyl is defined as above.

The term “arylthio,” alone or in combination, refers to an aryl thio radical, aryl-S-, wherein the term aryl is defined as below.

The term “aryl,” alone or in combination, refers to an optionally substituted aromatic ring system. The term aryl includes monocyclic aromatic rings, polyaromatc rings and polycyclic aromatic ring systems containing from six to about twenty carbon atoms. The term aryl also includes monocyclic aromatic rings, polyaromatic rings and polycyclic ring systems containing from 6 to about 12 carbon atoms (e.g., C₆, C₇, C₈, C₉, C₁₀, C₁₁, C₁₂), as well as those containing from 6 to about 10 carbon atoms (e.g., C₆, C₇, C₈, C₉, C₁₀). The polyaromatic and polycyclic aromatic rings systems may contain from two to four rings. Examples of aryl groups include, without limitation, phenyl, biphenyl, naphthyl and anthryl ring systems.

The term “heteroaryl” refers to optionally substituted aromatic ring systems containing from about five to about 20 skeletal ring atoms and having one or more heteroatoms such as, for example, oxygen, nitrogen, sulfur, and phosphorus. The term heteroaryl also includes optionally substituted aromatic ring systems having from 5 to about 12 skeletal ring atoms, as well as those having from 5 to about 10 skeletal ring atoms. The term heteroaryl may include five- or six-membered heterocyclic rings, polycyclic heteroaromatic ring systems and polyheteroaromatic ring systems where the ring system has two, three or four rings. The terms heterocyclic, polycyclic heteroaromatic and polyheteroaromatic include ring systems containing optionally substituted heteroaromatic rings having more than one heteroatom as described above (e.g., a six membered ring with two nitrogens), including polyheterocyclic ring systems of from two to four rings. The term heteroaryl includes ring systems such as, for example, furanyl, benzofuranyl, chromenyl, pyridyl, pyrrolyl, indolyl, quinolinyl, N-alkyl pyrrolyi, pyridyl-N-oxide, pyrimidoyl, pyrazinyl, imidazolyl, pyrazolyl, oxazolyl, benzothiophenyl, purinyl, indolizinyl, thienyl and the like.

The term “heteroarylalkyl” refers to a C₁-C₄ alkyl group (e.g., C₁, C₂, C₃, C₄) containing a heteroaryl group, each of which may be optionally substituted.

The term “heteroarylthio” refers to the group —S-heteroaryl.

The term “acyloxy” refers to the ester group —OC(O)—R, where R is H, alkyl, alkenyl, alkynyl, aryl, or arylalkyl, wherein the alkyl, alkenyl, alkynyl and arylalkyl groups may be optionally substituted.

The term “carboxy esters” refers to —C(O)OR where R is alkyl, aryl or arylalkyl, wherein the alkyl, aryl and arylalkyl groups may be optionally substituted. The term “carboxamido” refers to:

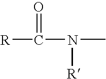

wherein each of R and R′ are independently selected from the group consisting of H, alkyl, aryl and arylalkyl, wherein the alkyl, aryl and arylalkyl groups may be optionally substituted.

The term “arylalkyl,” alone or in combination, refers to an alkyl radical as defined above in which one H atom is replaced by an aryl radical as defined above, such as, for example, benzyl, 2-phenylethyl and the like.

The terms “haloalkyl”, “haloalkenyl”, “haloalkynyl” and “haloalkoxy” include alkyl, alkenyl, alkynyl and alkoxy structures, as described above, that are substituted with one or more fluorines, chlorines, bromines or iodines, or with combinations thereof.

The terms “cycloalkyl”, “aryl”, “arylalkyl”, “heteroaryl”, “alkyl”, “alkynyl”, “alkenyl”, “haloalkyl” and “heteroalkyl” include optionally substituted cycloalkyl, aryl, arylalkyl, heteroaryl, alkyl, alkynyl, alkenyl, haloalkyl and heteroalkyl groups.

The term “carbocycle” includes optionally substituted, saturated or unsaturated, three- to eight-membered cyclic structures in which all of the skeletal atoms are carbon.

The term “membered ring” can embrace any cyclic structure, including 20 carbocycles and heterocycles as described above. The term “membered” is meant to denote the number of skeletal atoms that constitute the ring. Thus, for example, pyridine, pyran, and thiopyran are 6-membered rings and pyrrole, furan, and thiophene are 5-membered rings.

The term “acyl” includes alkyl, aryl, heteroaryl, arylalkyl or heteroarylalkyl substituents attached to a compound via a carbonyl functionality (e.g., —CO—alkyl, —CO-aryl, —CO—arylalkyl or —CO—heteroarylalkyl, etc.).

The term “alkylacylamino” as used herein refers to an alkyl radical appended to an acylamino group.

The term “acylamino” as used herein refers to an acyl radical appended to an amino group.

The term “substituted heterocycle” or heterocyclic group” as used herein refers to any 3-, or 4-membered ring containing a heteroatom selected from nitrogen, oxygen, phosphorus and sulfur or a 5- or 6-membered ring containing from one to three heteroatoms selected from the group consisting of nitrogen, oxygen, phosphorus and sulfur; wherein the 5-membered ring has 0-2 double bounds and the 6-membered ring has 0-3 double bounds; wherein the nitrogen and sulfur atom maybe optionally oxidized; wherein the nitrogen heteroatoms may be optionally quaternized; and including any bicyclic group in which any of the above heterocyclic rings is fused to a benzene ring or another 5- or 6-membered heterocyclic ring independently defined above. Heterocyclics can be unsubstituted or monosubstituted or disubstituted with substituents independently selected from hydroxy, halo, oxo (C═O), alkylimino (R—N═ wherein R is a alkyl group), amino, alkylamino, dialkylamino, acylaminoalkyl, alkoxy, thioalkoxy, polyalkoxy, alkyl, cycloalkyl or haloalkyl. Examples of heterocyclics include: imidazolyl, pyridyl, piperazinyl, azetidinyl, thiazolyl and triazolyl.

The term “glycosyl” as used herein refers to any pyranose or furanose saccharide group, including but not limited to D- or L-glucosyl, galactosyl, mannosyl, fucosyl, N-acetylneuraminyl, glucosaminyl, galactosaminyl, etc.

The term “disaccharide” as used herein refers to any pyranose or furanose saccharide group, including but not limited to D- or L-glucosyl, galactosyl, mannosyl, fucosyl, N-acetylneuraminyl, glucosaminyl, galactosaminyl, etc. linked through a glycosidic bond to another pyranose or furanose saccharide.

The term “oligosaccharide” as used herein refers to any pyranose or furanose groups including but not limited to D- or L-glucosyl, galactosyl, mannosyl, fucosyl, N-acetylneuraminyl, glucosaminyl, galactosaminyl, etc. linked through glycosidic bonds to another pyranose or furanose saccharides in which the number of saccharide groups is no less than three.

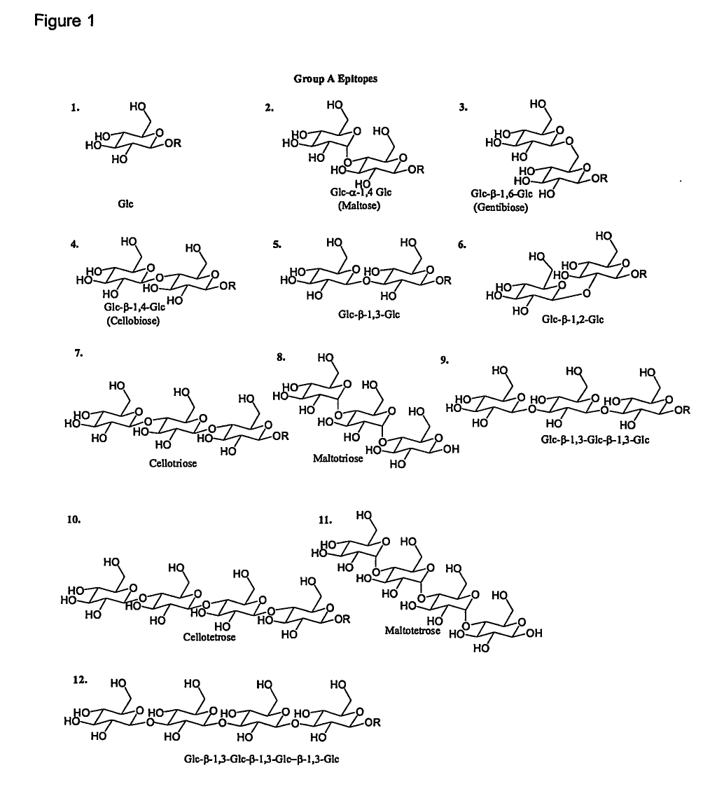

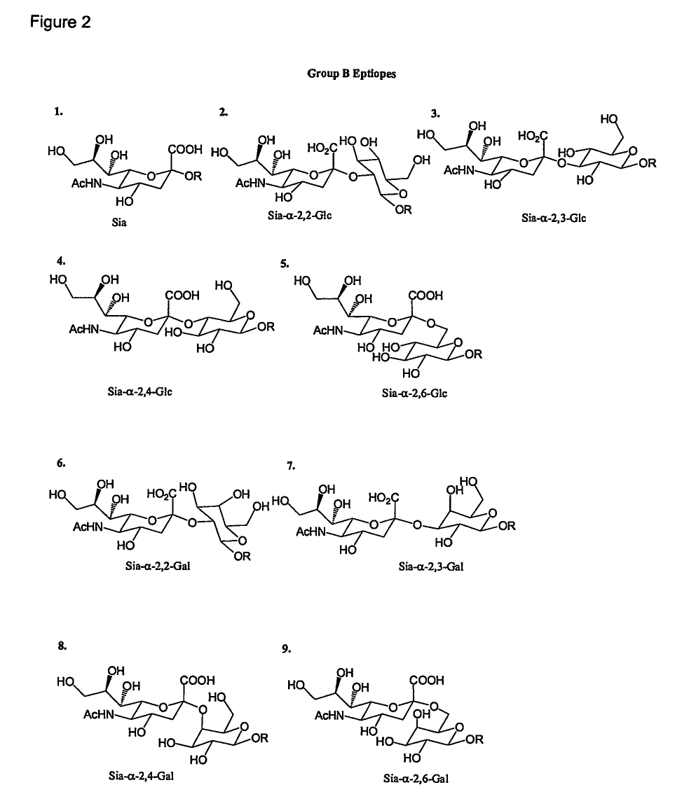

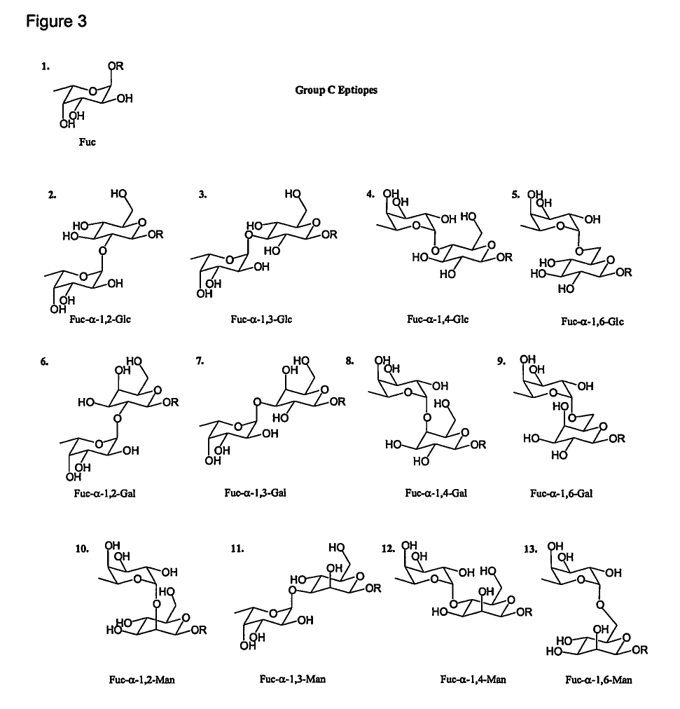

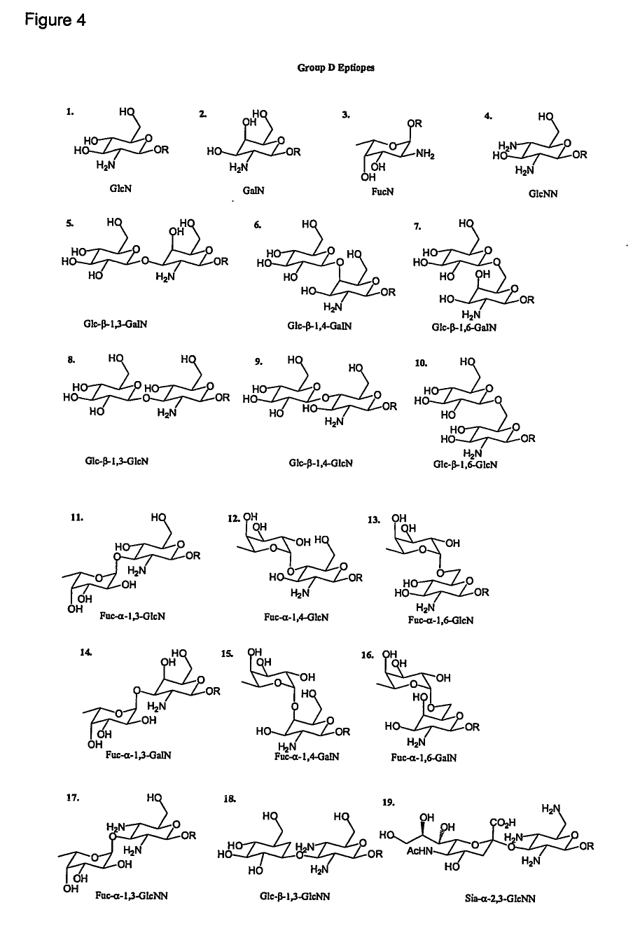

The term “glycosyl donor” as used herein refers to any pyranose or furanose saccharide or disaccharide group capable of glycosylating an acceptor such as hydroxyl, donors and includes but is not limited to suitably protected D- or L-thiotoluoyl glucopyranoside, thiotoluoyl galactopyranoside, mannopyranoside, fucopyranoside, N-acetylneuraminopyranoside, glucosaminopyranoside, galactosaminopyranoside, etc. The glycosidic linkages can be alpha, beta or alpha/beta mixtures. FIGS. 1 through 4 are examples of such saccharide and disaccharide groups.

The term “carbohydrate-activating group” as used herein refers to classes of functional groups that when attached to carbohydrates convert then into glycosyl donors. The carbohydrate-activating group is generally located at the anomeric position of the carbohydrate. Activating groups based on the type of anomeric functional group and their activating methods include but are not limited to: glycosyl halides, thioglycosides, 1-O-Acyl sugars, 1-O- and S-carbonates, trichloroimidates, etc.

The term “saccharide group” refers to an oxidized, reduced or substituted saccharide monoradical covalently attached via any atom of the saccharide moiety, preferably via the anomeric carbon atom. A saccharide refers to a carbohydrate which is a polyhydroxy aldehyde or ketone, or derivative thereof, having the empirical formula (CH₂O) wherein n is a whole integer, typically greater than 3. Monosaccharides, or simple sugars, consist of a single polyhydroxy aldehyde or ketone unit. Representative monosaccharides include, by way of illustration only, hexoses such as D-glucose, D-mannose, D-xylose, D-galactose, L-fucose, and the like; pentoses such as D-ribose or D-arabinose and ketoses such as D-ribulose or D-fructose. Disaccharides contain two monosaccharide units joined by a glycosidic linkage. Disaccharides include, for example, sucrose, lactose, maltose, cellobiose, and the like. Oligosaccharides typically contain from 3 to 10 monosaccharide units joined by glycosidic linkages. Polysaccharides (glycans) typically contain more than 10 such units and include, but are not limited to, molecules such as heparin, heparan sulfate, chondroitin sulfate, dermatan sulfate and polysaccharide derivatives thereof.

The term “sugar” generally refers to mono-, di- or oligosaccharides. A saccharide may be substituted, for example, glucosamine, galactosamine, acetylglucose, acetylgalactose, N-acetylglucosamine, N-acetyl-galactosamine, galactosyl-N-acetylglucosamine, N-acetylneuraminic acid (sialic acid), etc., and may contain sulfated and phosphorylated sugars. For the purposes of this definition, the saccharides can be either in their open or preferably in their pyranose form.

The term “sugar motif” generally refers to structures varying from natural neutral- , amino- , and acidic-sugars to those with five- and six-membered sugar frameworks with a wide variety of functional groups, some of which may be combinatorially generated.

The terms “stable surrogate” and “peptide surrogate” generally refer to structures varying from natural neutral - , amino - , and acidic-sugars to those with five - and six-membered sugar frameworks with a wide variety of functional groups, wherein said structures can replace acidic, basic, and neutral amino acids. Specifically, the introduced sugar motif can replace acidic amino acid residues if the sugar motif contains an acidic functional group, replace basic amino acid residues if the sugar motif contains a basic functional group, or replace neutral amino acids if the sugar motif contains a neutral functonal group.

The term “amino-containing saccharide group” refers to a saccharide group having at least one amino substituent. Representative amino-containing saccharides include mycaminose, desosamine, L-vancosamine, 3-desmethyl-vancosamine, 3-epi-vancosamine, 4-epi-vancosamine, acosamine, actinosamine, daunosamine, 3-epi-daunosamine, ristosamine, N-methyl-D-glucamine and the like.

“Optionally substituted” groups may be substituted or unsubstituted. The substituents of an “optionally substituted” group may include, without limitation, one or more substituents independently selected from the following groups or designated subsets thereof: alkyl, alkenyl, alkynyl, heteroalkyl, haloalkyl, haloalkenyl, haloalkynyl, cycloalkyl, aryl, heteroaryl, arylalkyl, heteroarylalkyl, alkoxy, aryloxy, haloalkoxy, amino, alkylamino, dialkylamino, alkylthio, arylthio, heteroarylthio, oxo, carboxyesters, carboxamido, acyloxy, H, F, Cl, Br, I, CN, NO₂, NH₂, N₃, NHCH₃, N(CH₃)₂, SH, SCH₃, OH, OCH₃, OCF₃, CH₃, CF₃, C(O)CH₃, CO₂CH₃, CO₂H, C(O)NH₂, pyridinyl, thiophene, furanyl, indole, indazol, esters, amides, phosphonates, phosphates, phosphoramides, sulfonates, sulfates, sulfonamides, carbamates, ureas, thioureas, thioamides, thioalkyls. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The term “halogen” includes F, Cl, Br and I.

The term “protected amino”, “amine protecting group” and “protected aminomethyl” as used herein refers to known amine protecting groups used in the synthetic organic chemistry art and include but are not limited to t-butoxycarbonyl (BOC), benzyloxycarbonyl (Cbz), azide (N₃), 2-trimethylsilylethoxycarbonyl (Teoc), allyloxycarbonyl (Alloc), 9-fluorenylmethyloxycarbonyl (Fmoc), acyl groups, such as formyl, acetyl, trihaloacetyl, benzoyl, and nitrophenylacetyl, sulfonamide groups, imine- and cyclic imide groups. Further examples of protected amino groups are described by Greene and Wuts in Protective Groups in Organic Synthesis, 2^{nd} edition (John Wiley & Sons, New York, 1991).

The term “modified amino” as used herein includes the terms “protected amino,” “amine protecting group,” “alkylacylamino,” “acylamino”and “carboxamido”.

The term “modified hydroxyl” as used herein includes the terms “protected hydroxyl”, “hydroxyl protecting group”, “protected hydroxymethyl,” “alkoxy,” “aryloxy,” “acyl,” “carboxy esters,” and “acyloxy”

The term “protected hydroxyl”, “hydroxyl protecting group”and “protected hydroxymethyl”as used herein refers to known hydroxyl protecting groups used in the synthetic organic chemistry art and include but are not limited to methoxymethyl (MOM), benzyloxymethyl (BOM), benzyl (Bn), Allyl (All), p-methoxybenzyl (PMB), t-butyldimethylsilyl (TBDMS), ester groups, such as acetate (Ac), chloroacetate and benzoate (Bz). Further examples of protected hydroxyl groups are descnbed by Greene and Wuts in Protective Groups in Organic Synthesis, 2^{nd} edition (John Wiley & Sons, New York, 1991).

The term “dimer half-life” generally refers to the time required for half of a given dimer to revert back to its two identical component molecules.

In accordance with the present invention, conjugation of the carbohydrate to the peptide is achieved in one of three different ways. First, conjugation is achieved through a linker attached at the anomeric position of suitably protected or unprotected carbohydrate residues and terminating in a carboxylic acid function. Second, conjugation is achieved through a linker attached at the anomeric position of such carbohydrate residues terminating in an amino function. Or third, conjugation is achieved through a direct glycosylation reaction at the anomeric position of such a carbohydrate moiety in which the anomeric position is suitably activated. In a preferred embodiment of the present invention, the carbohydrate can be coupled to a suitably protected peptide via a dehydration reaction at the α-amino terminus thus:

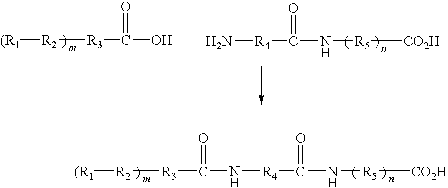

where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, silylation and the like;

R₂ is the atom or group at the anomeric position of the carbohydrate R₁ and may be O, S, NH or CH₂;

R₃ is a linker composed of alone or in any combination alkyl, alkenyl, alkynyl, heteroalkyl, heteroalkenyl, heteroalkynyl, alkoxy, aryloxy, alkylthio, arylthio, aryl, heteroaryl, heteroarylalkyl, heteroarylthio, acyloxy, carboxyesters, carboxamido, arylalkyl; haloalkyl, haloalkenyl, haloalkynyl, haloalkoxy, cycloalkyl, acyl, alkylacylamino or acylamino groups or amino acid residues;

R₄ and R₅, when substituted with NH₂ and CO₂H, are any natural amino acid or amino acid surrogate in which any reactive groups are suitably protected;

m is 1, 2, or 3; and n is any integer from 1 to about 100, but may be greater.

In another preferred embodiment of the present invention, one or more protected carbohydrates are conjugated through a linker to hydroxyl or amine functions on the side chains of amino acids along the backbone of a suitably protected peptide via a dehydration reaction thus:

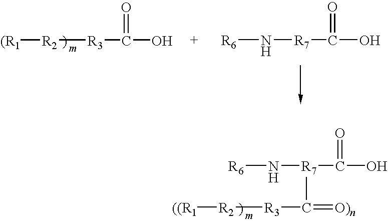

where R₁ is any carbohydrate including mono-, di-, tn-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, silylation and the like;

R₂ is the atom or group at the anomenic position of the carbohydrate R₁ and maybe O, S, NH or CH₂;

R₃ is a linker composed of alone or in any combination alkyl, alkenyl, alkynyl, heteroalkyl, heteroalkenyl, heteroalkynyl, alkoxy, aryloxy, alkylthio, aryithio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;

R₆ is a protecting group for an amine including but not limited to Fmoc, Boc, Cbz and the like;

R₇, when substituted with NH2 and CO2H, is any suitably protected natural or synthetic peptide containing one or more amino acid residues with side chains bearing a hydroxyl or amine function such as serine, threonine, hydroxyproline, tyrosine, lysine, hydroxylysine, arginine, or any other amino acid surrogates containing a hydroxyl or amine function on the side chain;

m is 1, 2, or 3; and n is any integer from 1 to about 100, but may be greater.

In another preferred embodiment of the present invention, the carbohydrate can be coupled to a suitably protected peptide via a dehydration reaction at the α-carboxyl terminus thus:

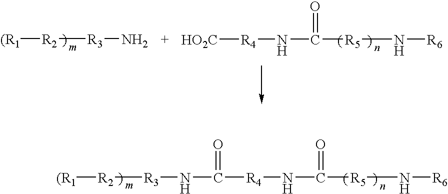

where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylabon, silylation and the like;

R₂ is the atom or group at the anomeric position of the carbohydrate R₁ and may be O, S, NH or CH₂;

R₃ is a linker composed of alone or in any combination alkyl, alkenyl, alkynyl, heteroalkyl, heteroalkenyl, heteroalkynyl, alkoxy, aryloxy, alkylthio, arylthio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;

R₄ and R₅, when substituted with NH₂ and CO₂H, are any natural amino acid or amino acid surrogate in which any reactive groups are suitably protected;

R₆ is a protecting group for an amine including but not limited to Fmoc, Boc, Cbz and the like;

m is 1, 2, or 3; and n is any integer from 1 to about 100, but may be greater.

In another preferred embodiment of the present invention, one or more protected carbohydrates are conjugated through a linker to carboxyl functions on the side chains of amino acids along the backbone of a suitably protected peptide via a dehydration reaction thus:

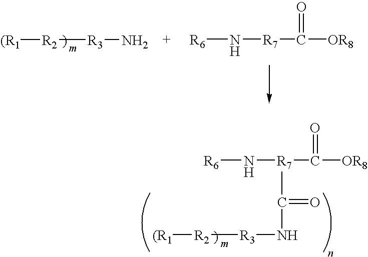

where R₁ is any carbohydrate including mono-, di-, tn-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, silylation and the like;

R₂ is the atom or group at the anomenic position of the carbohydrate R₁ and maybe O, S, NH or CH₂;

R₃ is a linker composed of alone or in any combination alkyl, alkenyl, alkynyl, heteroalkyl, heteroalkenyl, heteroalkynyl, alkoxy, aryloxy, alkylthio, aryithio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;

R₆ is a protecting group for an amine including but not limited to Fmoc, Boc, Cbz and the like;

R₇, when substituted with NH2 and CO2H, is any suitably protected natural or synthetic peptide containing one or more amino acid residues with side chains bearing a carboxyl function such as aspartic acid or glutamic acid, or any other amino acid surrogates containing a carboxyl function on the side chain;

R₈ is a protecting group for a carboxyllc acid including but not limited to methyl, ethyl, t-butyl, allyl, benzyl, succinyl, trimethylsilylethyl, p-nitrophenyl, pentafluorophenyl, and the like;

m is 1, 2, or 3; and n is any integer from 1 to about 100, but may be greater.

In another preferred embodiment of the present invention, a protected carbohydrate is conjugated to a hydroxy or amino group on the side chain of an amino acid along the backbone of a suitably protected peptide via direct glycosylation thus:

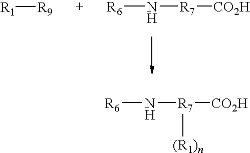

where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, silylation and the like;

R₆ is a protecting group for an amine including but not limited to Fmoc, Boc, Cbz and the like;

R₇, when substituted with NH₂ and CO₂H, is any suitably protected natural or synthetic peptide containing one or more amino acid residues with side chains bearing a hydroxyl or amine function such as serine, threonine, hydroxyproline, tyrosine, lysine, hydroxylysine, arginine, or any other amino acid surrogates containing a hydroxyl or amine function on the side chain;

R₉ is a sugar activating group such as but not limited to sulfide, trichloroacetimidate, bromide, chloride, fluoride, acyloxy, sulfoxide, phosphite and the like;

and n is any integer from 1 to about 100, but may be greater.

Thus, with respect to the above described preferred embodiments, the present invention encompasses both the method of making such glycoconjugates as well as the resulting glycoconjugate compounds and compositions.

### Methods

Carbohydrates including mono-, di-, tn- and tetrasaccharides and larger are prepared using OPopS™ technology (WO000/09527) in a one-pot fashion in which the final acceptor contains a carboxylic ester or a protected amine. Following this glycosylation step the protecting group can be removed allowing for coupling to the peptide. Those of skill in the art recognize that the present invention is not limited by these coupling methods. Rather, those of skill in the art recognize that the present invention includes other available coupling methods.

For example, for direct attachment of a sugar motif to a hydroxyl group of the peptide through its anomeric position, the sugar donor portion may have an anomeric activating group including but not limited to alkyl or aryl thio, halide (Br, Cl, F), trialkyl or tniaryl phosphate, dialkyl or diaryl phosphite, imidate (trichloroacetimidate), OH, or O-acyl group. An activating reagent can be used to promote the condensation reaction between the sugar donor and the peptide acceptor, and the reagents include but are not limited to Lewis acids (trialkylsilyl trifiate, boron trifluoride-etherate), methyl trifiate, dimethyl(methylthio)sulfonium triflate, N-iodosuccinimide (NIS), N-bromosuccinimide (NBS), NBS/trifiuoromethanesulfonic acid (triflic acid), NBS/triflic acid, NIS/trialkylsilyl triflate, NBS/trialkylsilyl trifiate, NBS/tetraalkylammonium trifluoromethansulfonate, NBS/tetraalkylammonium trifluoromethansulfonate, 1-benzenesulfinyl piperidine/triflic anhydride and NBS/trimethylsilyl triflate, SnCl₂/AgClO₄, SnCl₂/TrClO₄, SnCI2/AgOTf, TMSOTf, SiF₄, Cp₂MCl₂/AgClO₄ (M=Zr or Hf), Cp₂ZrCl₂/AgBF₄, Cp₂HfCl₂/AgOTf, Yb(OTf)₃, La(ClO₄)₃, La(ClO₄)₃/Sn(OTf)₂, Nafion-H, montmorillonite K-10, and TrB(C₆F₅)₄. (References: Toshima, K.; Tatsuta, K. Chemical Review, 1993, 93, 1503-1531; Boons, G.-J. Tetrahedron, 1996, 52, 1095-1121; Garegg, P. J. Adv. Carbohydr. Chem. Biochem. 1997, 52, 179-205; Toshima, K. Carbohydr. Res. 2000, 327, 15-26).

The peptide can be prepared using solid phase synthesis techniques (Merrifleld, R. B., J. Am. Chem. Soc., 1963, 85, 2149-2154) to give a molecule in which one or more amino, hydroxyl or carboxyl groups remain unprotected. These may be the N-terminal ct-amine, the C-terminal c-carboxylic acid or amines, alcohols or carboxylic acids on side chains of amino acids, which may be serine, threonine, hydroxyproline, tyrosine, lysine, hydroxylysine, arginine, aspartic acid or glutamic acid or any other amino acid surrogate bearing a hydroxyl, amino, or carboxyl group on the side chain.

The reaction between the carboxylic acid or amine group on the carbohydrate linker and the peptide can be promoted with the use of a coupling agent such as, 2-(1H-9-azobenzotriazole-1-yl)-1,1,3,3-tetramethyluronium hexafluorophosphate (HATU); 2-(1H-benzotriazole-1-yl-1,1,3,3-tetramethyluronium hexafluorophosphate (HBTU); 2-(1H-benzotriazole-1-yl)-1,1,3,3-tetramethyluronium tetrafluoroborate (TBTU); benzotriazole-1-yl-oxy-tris(dimethylamino)phosphonium hexafluorophosphate (BOP); benzotriazole-1-yl-oxy-trispyrrolidinophosphonium hexafluorophosphate (PyBOP); 1-ethyl-3-[3-(dimethylamino)propyl]carbodiimide (EDCI); N—N′-dicyclohexylcarbodiimide (DCC) and the like.

Alternatively the peptide may be directly glycosylated with one or more carbohydrate moieties at the side chain hydroxyl or amino group of an amino acid, which may be serine, threonine, hydroxyproline, tyrosine, lysine, proline or arginine or any other amino acid surrogate bearing a hydroxyl or amino group on the side chain. Glycosidic bond formation may be achieved through the use of OPopS™ technology (WO000/09527) in a one-pot fashion in which the final acceptor added to the reaction is a peptide containing a free hydroxyl or amino group.

Global deprotection of the glycoconjugate using reagents and conditions well known to one skilled in the art affords a new class of biologically active peptides whose half-lives may be determined through assays with animal tissue homogenate or plasma or commercially available enzymes known to degrade peptides such as but not limited to trypsin, chymotrypsin, alanyl aminopeptidase, lysine aminopeptidase, leucine aminopeptidase and prolyl carboxypeptidase and the like. The half-lives may be measured using a variety of analytical techniques such as mass spectrometry, gas chromatography, high performance liquid chromatography, gas chromatography/mass spectrometry or liquid chromatography/mass spectrometry. By comparison of the half-life of a glycopeptide with that of the native peptide from which it was derived the effect of glycoconjugation on the peptide's stability towards peptidase enzymes can be demonstrated.

The three-dimensional conformation of the glycopeptides of this present invention is substantially similar to their corresponding peptides which contain amino acids and no sugars. This three-dimensional structure can be determined by techniques commonly used in the art, such as circular dichroism (CD) spectroscopy and optical rotary dispersion (ORD).

### Pharmaceutical Formulation And Administration

Once isolated, glycopeptides (i.e. glycosylated peptides or analogs thereof) can be put in pharmaceutically acceptable formulations, such as those described in Remington's Pharmaceutical Sciences, 18th ed., Mack Publishing Co., Easton, Pa. (1990), incorporated by reference herein, and used for specific treatment of diseases and pathological conditions with little or no effect on healthy tissues. In a preferred embodiment, the composition is held within a container which includes a label stating to the effect that the composition is approved by the FDA in the United States (or other equivalent labels in other countries) for treating a disease or condition described herein. Such a container will provide therapeutically effective amount of the active ingredient to be administered to a host.

The particular glycopeptides that affect the disorders or conditions of interest can be administered to a patient either by themselves, or in pharmaceutical compositions where they are mixed with suitable carriers or excipient(s). In treating a patient exhibiting a disorder of interest, a therapeutically effective amount of an agent or agents such as those listed herein is administered. A therapeutically effective dose refers to that amount of the compound that results in amelioration of symptoms or a prolongation of survival in a patient.

The compounds also can be prepared as pharmaceutically acceptable salts. Examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. (See e.g., PCT/US92/03736). Such salts can be derived using acids such as hydrochloric acid, sulfuric acid, phosphoric acid, sulfamic acid, acetic acid; citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the compound, for example, to increase the solubility of the compound. Examples of carriers and excipients include calcium carbonate, calcium phosphate, various sugars or types of starch, cellulose derivatives, gelatin, vegetable oils, polyethylene glycols and physiologically compatible solvents. In addition, the molecules tested can be used to determine the structural features that enable them to act on the ob gene control region, and thus to select molecules useful in this invention. Those skilled in the art will know how to design drugs from lead molecules, using techniques such as those disclosed in PCT publication WO 94/18959, incorporated by reference herein.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any glycopeptide used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et-al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. Those skilled in the art can prepare injectable peptide and protein formulations as described in, Formulation and Delivery of Proteins and Peptides: design and Development Strategies in “Formulation and Delivery of Proteins and Peptides” Cleland, J. L. and Langer, R. eds., ACS Symposium Series 567, pp. #1-21, American Chemical Society, Washington, DC, 1994. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Those of skill in the art will recognize that the present invention comprises multiple improvements to characteristics of the glycosylated peptide including, but 30 not limited to enhancement of stability in the presence of peptidases and proteases, thermal stability, dimer half-life, pharmaceutical properties, bioavailability and/or plasma half-life.

Methods and materials are described herein. However, methods and materials similar or equivalent to those described herein can be also used to obtain variations of the present invention. The materials, methods, and examples are illustrative only and not intended to be limiting.

### EXAMPLES

### Example 1

Synthesis of 1: Peptide A (SEQ. ID NO. 1: Lys-Lys-Arg-Gly-Gly-Ser)(200 mg, 0.05 mmol loaded peptide) was treated with carbohydrate glycolic acid B (30 mg, 0.075 mmol), O-benzotriazol-1-yl-N,N,N′,N′-tetramethyluronium hexafluorophosphate (HBTU) (28 mg, 0.075 mmol) and diisopropylethylamine (DIPEA) (19 mg, 26 μL, 0.150 mmol) in 2 mL of DMF for 90 minutes. The conjugated peptide was cleaved from the resin by treatment with 95:2.5:2.5 TFA:TIS:H₂O for 90 minutes. The mixture was filtered and the filtrate was concentrated under reduced pressure to give crude glycopeptide 1. MS (ES⁺) m/z 799 (M+2H)²⁺ (C₆₅H₁₀₁N₁₁O₃₅) (Scheme 1).

Degradation Study: A solution of 1 (1 mg) in 0.1 M, pH 7.5 potassium phosphate buffer (1 mL) was treated with 120 units of trypsin (10 μL of a 1 mg/mL solution of trypsin 12000 units/mg). A 20 μL aliquot of the sample was immediately analyzed by LCMS.

LC conditions:

Column: Waters Symmetry 300, 4.6×250 mm, C18, 5 micron particle size.

Solvent A: H₂O/0.1 % TFA

Solvent B: Acetonitrile/0.1% TFA

Gradient: 15-40% B over 30 minutes; 15% B isocratic for 10 minutes

The initial injection was defined as “time 0” and one subsequent injection was carried out at 40 minutes. The degradation products were characterized by mass spectrometry using electrospray positive ionization (ES⁺). Product ratios were determined by integration of photodiode array (PDA) spectra. A possible degradation pathway is shown in Scheme 2 and the degradation data are summarized in Table 1.

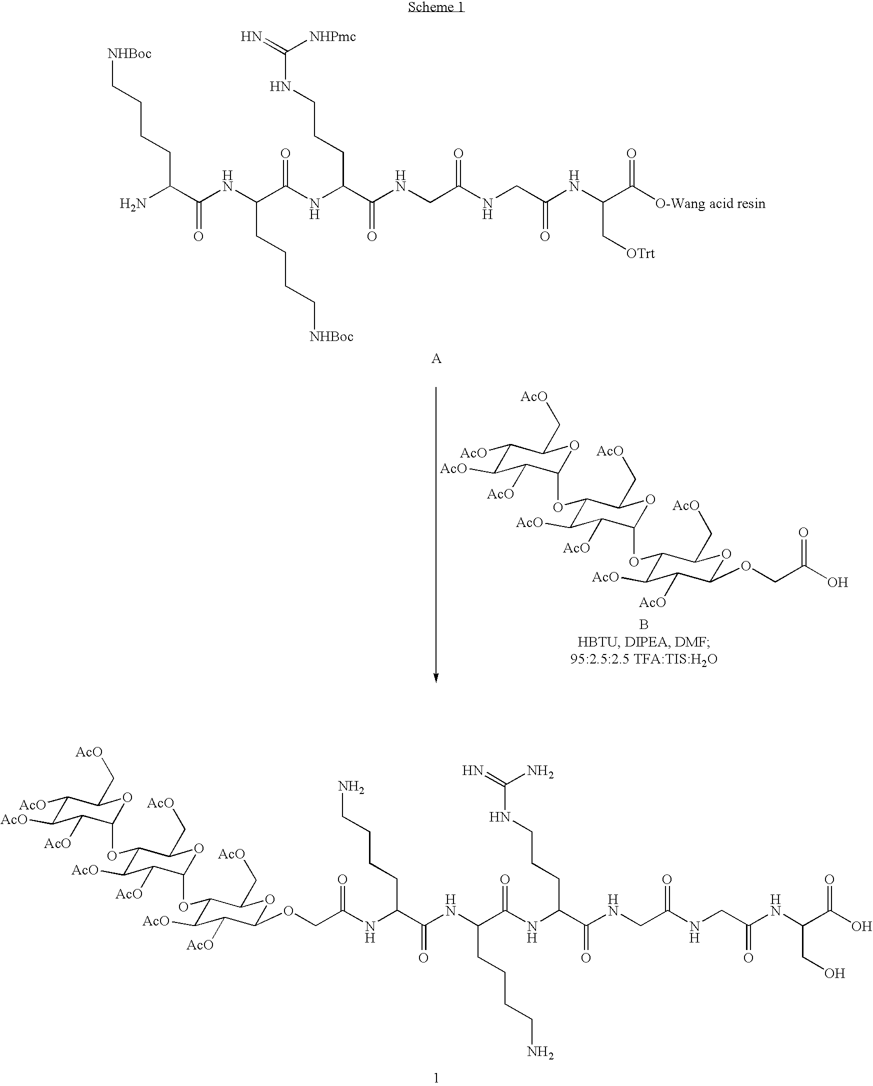

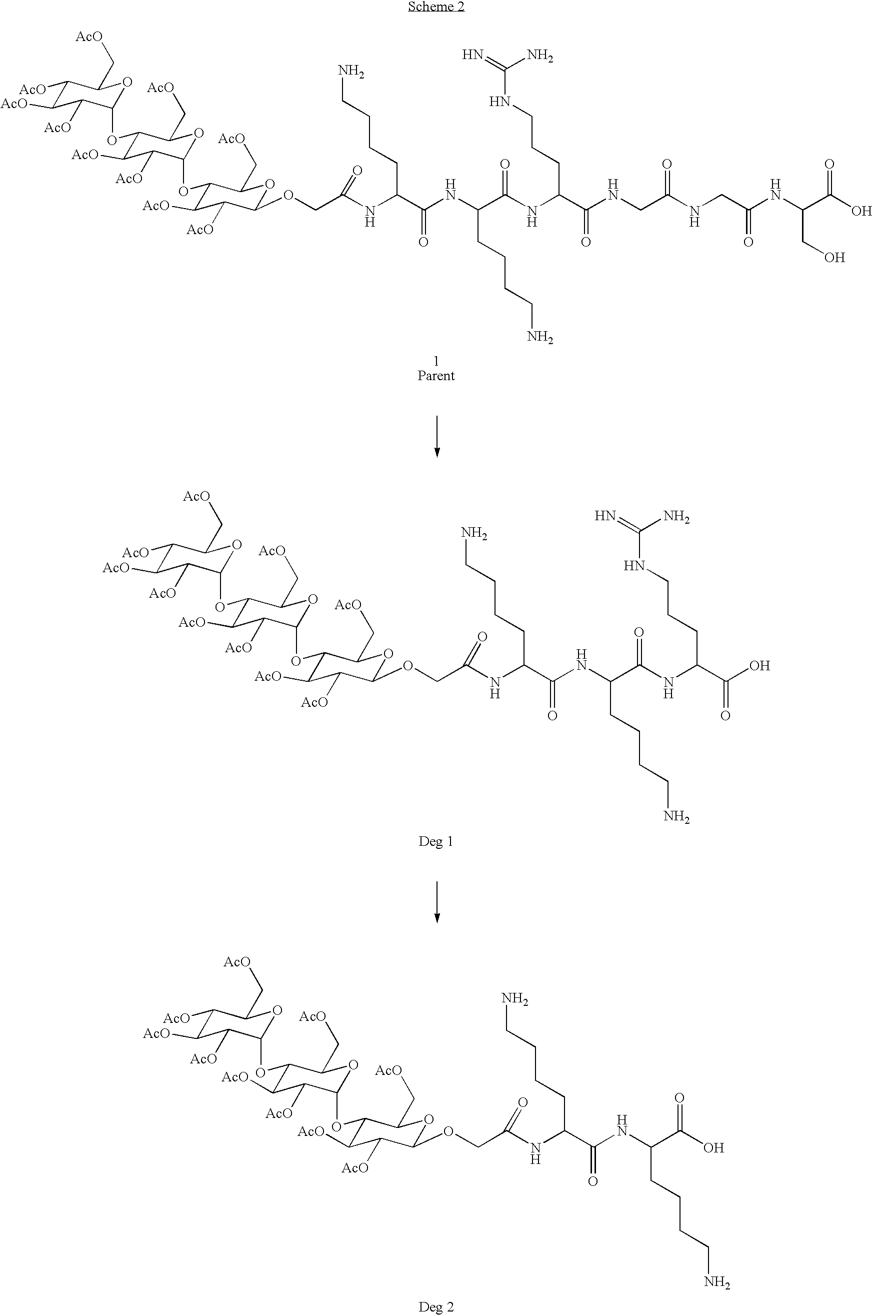

### EXAMPLE 2

Synthesis of 2: Prepared using the procedure described in Example 1 from peptide A (SEQ ID No. 1) and carbohydrate glycolic acid C to give the crude triphenylmethyl protected glycopeptide 2 (Scheme 3). MS (ES⁺) m/z 642 (M+H+Na)²⁺(C₆₀H₈₄N₁₂NaO₁₈).

Degradation study: Compound 2 was degraded using trypsin as described in Example 1 (Scheme 4). The data are summarized in Table 1.

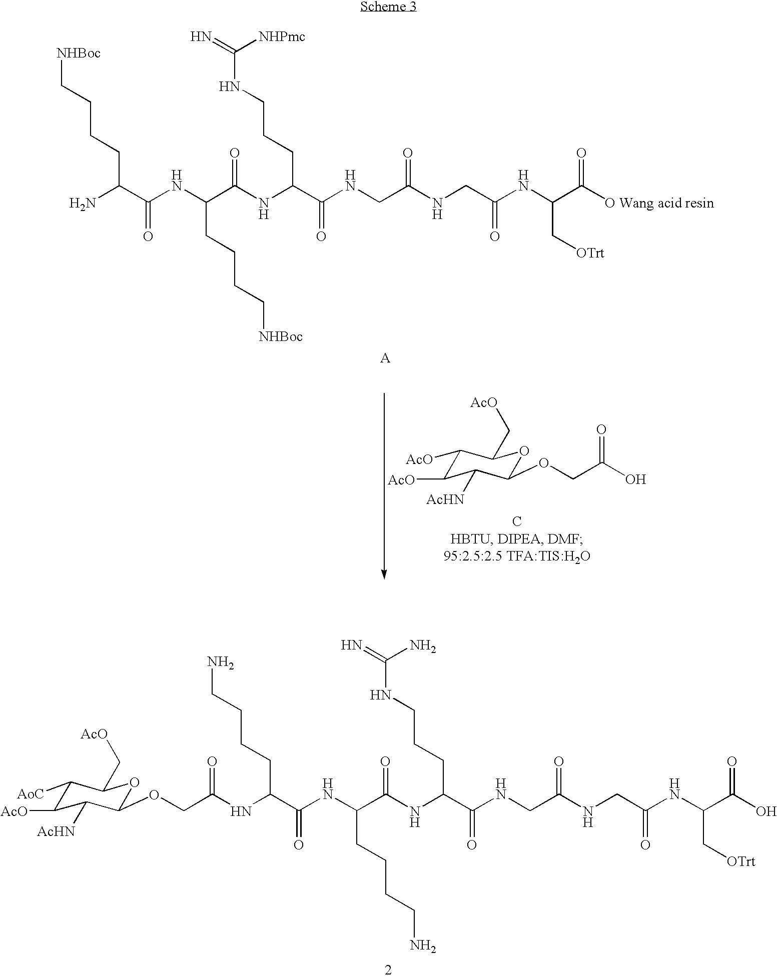

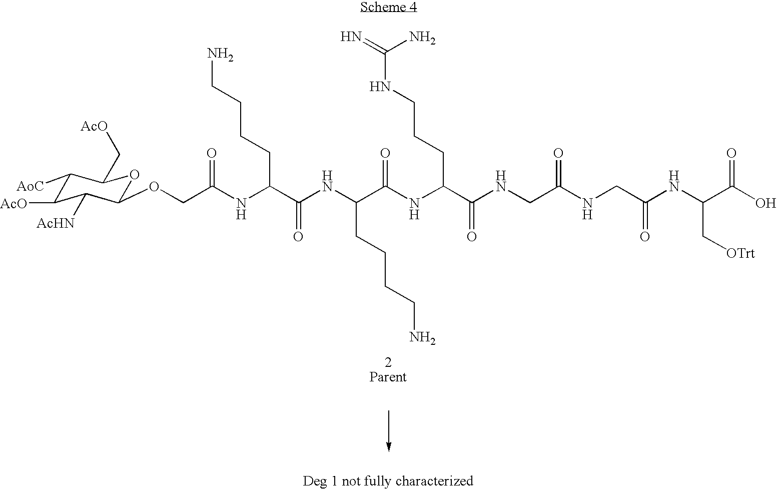

### Example 3

Synthesis of 3: Triphenylmethyl protected glycopeptide 2 was treated with 95:2.5:2.5 TFA:TIS:H₂O for 75 minutes and the mixture was concentrated to give crude 3 (Scheme 5). MS (ES⁺) m/z 510 (M+2H)²⁺ (C₄₁H₇₀N₁₂O₁₈).

Degradation Study: Compound 3 was degraded using trypsin as described in Example 1. Only the parent 3 and no degradation products were observed. The data are summarized in Table 1.

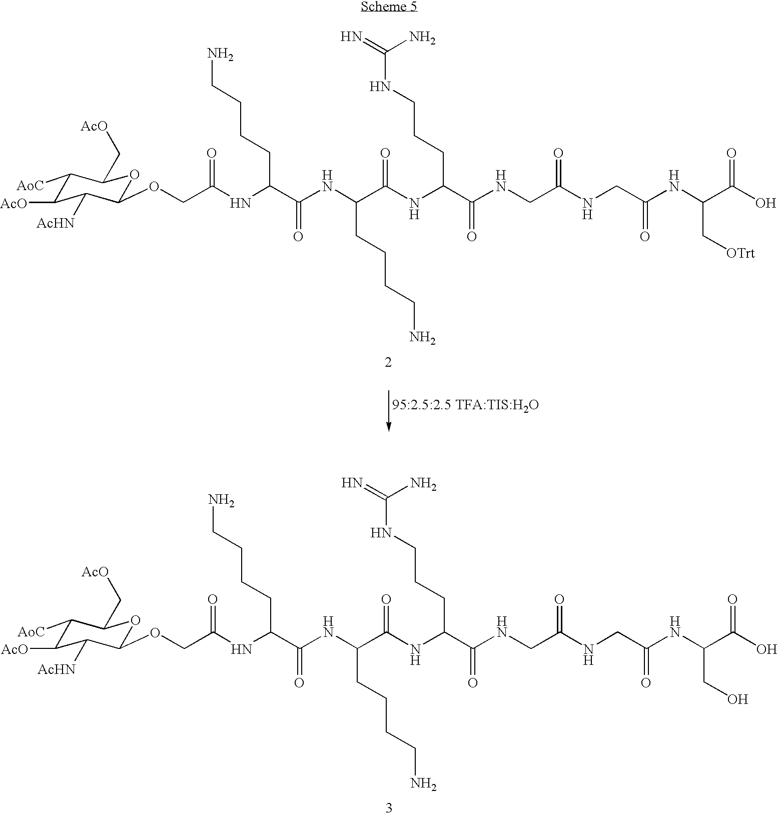

### EXAMPLE 4

Synthesis of 4. Peptide D (Protected Peptide A: SEQ ID No. 1)(200 mg) was treated with 95:2.5:2.5 TFA:TIS:H₂O for 90 minutes. The mixture was filtered and the filtrate was concentrated under reduced pressure to leave the crude flourenylmethyloxycarbonyl (Fmoc) protected peptide 4 (Scheme 6). MS (ES⁺) m/z 854 (M+H)⁺(C₄₀H₅₉N₁₁O₁₀).

Degradation Study: Compound 4 was degraded using trypsin as described in Example 1 (Scheme 7). The data are summarized in Table 1.

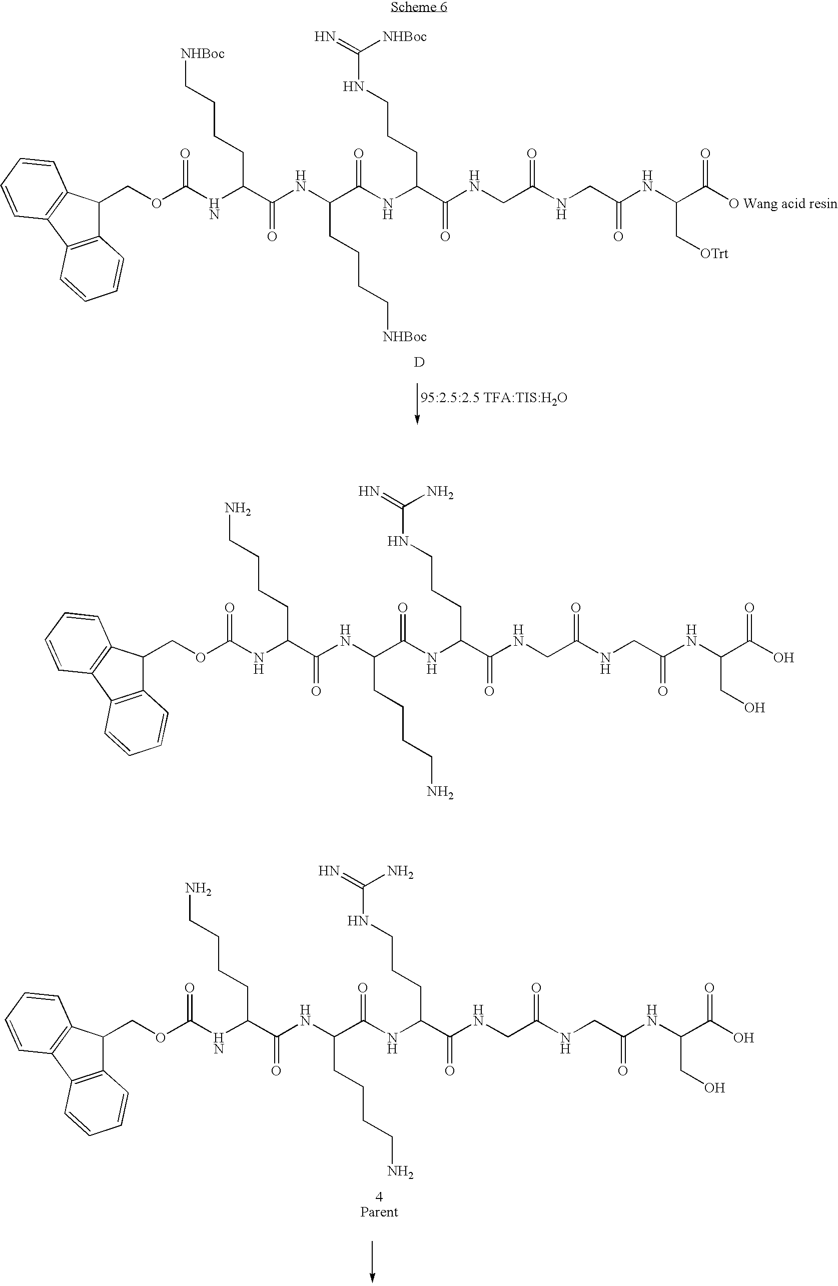

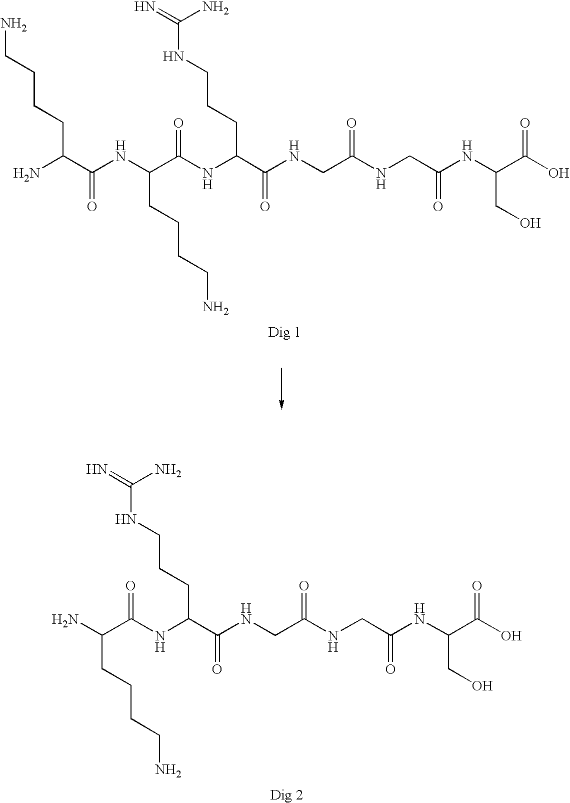

TABLE 1

| Compound | Parent (%) | Deg 1 | Deg 2 |
|---|---|---|---|
| 1 time 0 | 75 | 5 | 20 |
| 1 time 40 | 0 | 0 | 100 |
| 2 time 0 | 60 | 0 | ND |
| 2 time 40 | 27 | 67 | ND |
| 3 time 0 | 100 | ND | ND |
| 3 time 40 | 100 | ND | ND |
| 4 time 0 | 92 | 4 | 4 |
| 4 time 40 | 1 | 54 | 45 |

ND = not detected.

These data indicate different modes of enzymatic degradation for all 4 of the representative compounds. The presence of the carbohydrate moiety in 1 alters the cleavage site of trypsin as compared with 4, which is simply an fmoc-protected peptide. In the case of 4, hydrolysis occurs sequentially inward from the N-terminus. In the case of 1, hydrolysis is initially observed between the arginine and glycine residues. After 40 minutes, the two lysine residues conjugated to the carbohydrate were still observed, illustrating a markedly different reaction pathway than was observed for 4. The results for 2 indicate a single, as yet uncharacterized product of the enzyme reaction. We believe that the product is a stable peptide conjugate containing all 6 of the amino acid residues and the carbohydrate. A slight chemical modification or isomerization has occurred. Compound 3 shows remarkable stability to trypsin. No degradation products were observed after 40 minutes.

It is to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

We claim:

1. A compound selected from the group consisting of:
Compounds of Formula I and pharmaceutically acceptable salts, esters and prodrugs thereof: 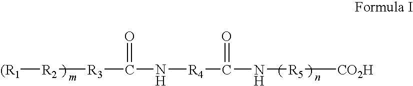
where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, and silylation;
R₂ is the atom or group at the anomeric position of the carbohydrate R₁ and may be O, S, NH or CH₂;
R₃ is a linker composed of alone or in any combination alkyl, alkenyl, alkynyl, heteroalkyl, heteroalkenyl, heteroalkynyl, alkoxy, aryloxy, alkylthio, arylthio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;
R₄ and R₅, when substituted with NH₂ and CO₂H, are any natural amino acid or amino acid surrogate;
m is 1, 2, or 3; and n is any integer from 1 to 200.

2. The compound according to claim 1, wherein n is any integer from 1 to 100.

3. The compound according to claim 1 having characteristics comprising:
increased stability in the presence of peptidases; increased stability in the presence of proteases; increased thermal stability; increased dimer half-life; increased bioavailability; and increased plasma half-life relative to a non-glycosylated analog of the compound.

4. The compound according to claim 1 wherein a particular sugar motif serves as a stable surrogate for a specific amino acid residue.

5. A compound selected from the group consisting of:
Compounds of Formula II and pharmaceutically acceptable salts, esters and prodrugs thereof: 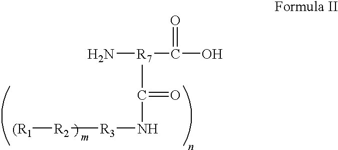
where R₁ is any carbohydrate including mono-, di-, tn-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, and silylation;
R₂ is the atom or group at the anomeric position of the carbohydrate R₁ and may be O, S, NH or CH₂;
R₃ is a linker composed of alone or in any combination alkyl, alkenyl, alkynyl, heteroalkyl, heteroalkenyl, heteroalkynyl, alkoxy, aryloxy, alkylthio, arylthio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;
R₇, when substituted with NH₂ and CO₂H, is any natural or synthetic peptide containing one or more amino acid residues with side chains bearing a hydroxyl or amine function such as serine, threonine, hydroxyproline, tyrosine, lysine, hydroxylysine, arginine, or any other amino acid surrogates containing a hydroxyl or amine function on the side chain;
m is 1, 2, or 3; and n is any integer from 1 to 200.

6. The compound according to claim 5, wherein n is any integer from 1 to 100.

7. The compound according to claim 5 having characteristics comprising:
increased stability in the presence of peptidases; increased stability in the presence of proteases; increased thermal stability; increased dimer half-life; increased bioavailability; and increased plasma half-life relative to a non-glycosylated analog of the compound.

8. The compound according to claim 5 wherein a particular sugar motif serves as a stable surrogate for a specific amino acid residue.

9. A compound selected from the group consisting of:
Compounds of Formula III and pharmaceutically acceptable salts, esters and prodrugs thereof: 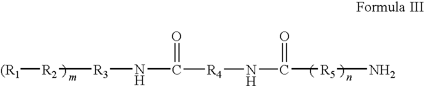
where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, and silylation;
R₂ is the atom or group at the anomeric position of the carbohydrate R₁ and may be O, S, NH or CH₂;
R₃ is a linker composed of alone or in any combination alkyl, alkenyl, alkynyl, heteroalkyl, heteroalkenyl, heteroalkynyl, alkoxy, aryloxy, alkylthio; arylthio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;
R₄ and R₅, when substituted with NH₂ and CO₂H, are any natural amino acid or amino acid surrogate;
m is 1, 2, or 3; and n is any integer from 1 to 200.

10. The compound according to claim 9, wherein n is any integer from 1 to 100.

11. The compound according to claim 9 having characteristics comprising: increased stability in the presence of peptidases; increased stability in the presence of proteases; increased thermal stability; increased dimer half-life; increased bioavailability; and increased plasma half-life relative to a non-glycosylated analog of the compound.

12. The compound according to claim 9 wherein a particular sugar motif serves as a stable surrogate for a specific amino acid residue.

13. A compound selected from the group consisting of:
Compounds of Formula IV and pharmaceutically acceptable salts, esters and prodrugs thereof: 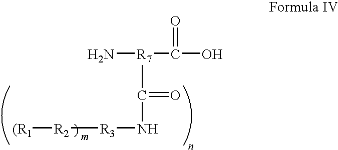
where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, and silylation;
R₂ is the atom or group at the anomeric position of the carbohydrate R₁ and maybe O, S, NH or CH₂;
R₃ is a linker composed of alone or in any combination alkyl, alkenyl, alkynyl, heteroalkyl, heteroalkenyl, heteroalkynyl, alkoxy, aryloxy, alkylthio, arylthio, aryl, heteroaryl, heteroarylalkyl, heteroarylthio, acyloxy, carboxyesters, carboxamido, arylalkyl, haloalkyl, haloalkenyl, haloalkynyl, haloalkoxy, cycloalkyl, acyl, alkylacylamino or acylamino groups or amino acid residues;
R₇, when substituted with NH₂ and CO₂H, is any natural or synthetic peptide containing one or more amino acid residues with side chains bearing a carboxyl function such as aspartic acid or glutamic acid, or any other amino acid surrogates containing a carboxyl function on the side chain;
m is 1, 2, or 3; and n is any integer from 1 to 200.

14. The compound according to claim 13, wherein n is any integer from 1 to 100.

15. The compound according to claim 13 having characteristics comprising: increased stability in the presence of peptidases; increased stability in the presence of proteases; increased thermal stability; increased dimer half-life;
increased bioavailability; and increased plasma half-life relative to a non-glycosylated analog of the compound.

16. The compound according to claim 13 wherein a particular sugar motif serves as a stable surrogate for a specific amino acid residue.

17. A compound selected from the group consisting of:
Compounds of Formula V and pharmaceutically acceptable salts, esters and prodrugs thereof: 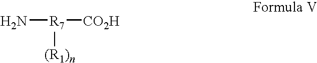
where R₁ is any carbohydrate including mono-, di-, tri-, and tetrasaccharides and larger, which may contain one or more amino sugars, deoxy sugars or sialic acid sugars in any combination and in which any hydroxyl, amino or carboxyl functions are suitably modified by sulfation, alkylation, acylation, deoxygenation, diazotization, pegylation, and silylation;
R₇, when substituted with NH₂ and CO₂H, is any natural or synthetic peptide containing one or more amino acid residues with side chains bearing a hydroxyl or amine function such as serine, threonine, hydroxyproline, tyrosine, lysine, hydroxylysine, arginine, or any other amino acid surrogates containing a hydroxyl or amine function on the side chain;
and n is any integer from 1 to 200.

18. The compound according to claim 17, wherein n is any integer from 1 to 100.

19. The compound according to claim 17 having characteristics comprising: increased stability in the presence of peptidases; increased stability in the presence of proteases; increased thermal stability; increased dimer half-life; increased bioavailability; and increased plasma half-life relative to a non-glycosylated analog of the compound.

20. The compound according to claim 17 wherein a particular sugar motif serves as a stable surrogate for a specific amino acid.

21. A method for producing the compound of claim 1 comprising reacting an α-amino group of a peptide molecule with a carboxylic acid group, joined through a linker or spacer to a carbohydrate moiety to yield a glycopeptide.

22. The method according to claim 21 wherein the stability of the glycopeptide towards peptidase enzymes is increased relative to the peptide.

23. A method for producing the compound of claim 5 comprising reacting an amine or hydroxyl group on a side chain of an amino acid within a peptide molecule with a carboxylic acid group, joined through a linker or spacer to a carbohydrate moiety to yield a glycopeptide.

24. The method according to claim 23 wherein the stability of the glycopeptide towards peptidase enzymes is increased relative to the peptide.

25. A method for producing the compound of claim 9 comprising reacting an α-carboxyl group of a peptide molecule with an amino group, joined through a linker or spacer to a carbohydrate moiety to yield a glycopeptide.

26. The method according to claim 25 wherein the stability of the glycopeptide towards peptidase enzymes is increased relative to the peptide.

27. A method of producing the compound of claim 13 comprising reacting a carboxyl group on a side chain of an amino acid within a peptide molecule with an amino group, joined through a linker or spacer to a carbohydrate moiety to yield a glycopeptide.

28. The method according to claim 27 wherein stability of the glycopeptide towards peptidase enzymes is increased relative to the peptide.

29. A method of producing the compound of claim 17 comprising glycosylating a hydroxyl or amino group on a side chain of an amino acid within a peptide molecule with a carbohydrate moiety activated at the anomeric position to yield a glycopeptide.

30. The method according to claim 29 wherein the stability of the glycopeptide towards peptidase enzymes is increased relative to the peptide.


SEQUENCE LISTING (1 sequence)

SEQ ID NO: 1 - protein, 6 aa, artificial sequence
  peptide
KKRGGS